(19) United States
(12) Patent Application Publication
(10) Pub. No.: US 2023/0211169 A1
(43) Pub. Date: Jul. 6, 2023

(54) ALZHEIMER'S DISEASE PREVENTION OR TREATMENT WITH LOW INTENSITY AND HIGH FREQUENCY MAGNETIC STIMULATION

(71) Applicant: Actipulse Neuroscience Inc., Cambridge, MA (US)

(72) Inventor: Alain Andre CHÂTILLON, Mexico City (MX)

(21) Appl. No.: 18/009,151

(22) PCT Filed: Jun. 9, 2021
(86) PCT No.: PCT/US2021/036597

§ 371 Date: Dec. 8, 2022

Related U.S. Application Data
(60) Provisional application No. 63/036,601, filed on Jun. 9, 2020.

(51) Int. Cl. A61N 2/00; A61N 2/02

(52) U.S. Cl. CPC A61N 2/006; A61N 2/02

(57) ABSTRACT

The present disclosure provides, in part, methods of treatment for Alzheimer’s disease comprising applying repetitive transcranial magnetic stimulation (rTMS) therapy to a patient in need thereof. The present disclosure also provides devices that generate a low intensity pulsed magnetic field and variable frequencies.

### CROSS-REFERENCE TO RELATED APPLICATION

This application claims the benefit of U.S. Provisional Pat. Application No. 62/036,601, filed Jun. 9, 2020, the entire contents of which are herein incorporated by reference.

### FIELD

The present disclosure provides, in part, clinical applications of a non-invasive brain stimulation, e.g. repetitive Transcranial Magnetic Stimulation (rTMS), as it pertains to neuromodulation in the treatment of neurological disorders and diseases.

### BACKGROUND

Alzheimer’s disease (AD) is a progressive disorder that causes brain cells to degenerate and die. “Mild cognitive impairment” (MCI) is a progressive disease that is diagnosed when loss of memory and critical thinking skills are beyond what would be expected for an individual given their age and educational background; but the loss is not so severe as to merit a diagnosis of dementia. MCI is thought to be a transitional state between normal aging and AD. The conversion rate from MCI to AD is estimated to be approximately 10% per year and that number increases every year.

Currently, there is no treatment that can reverse or stop the neural damage caused by Alzheimer’s disease. Multiple treatment options have been studied without significant success. Transcranial magnetic stimulation (TMS) is a non-invasive, painless neurostimulation technique with high safety profile that has been used as a neurologic and psychiatric treatment. However, the use of repetitive TMS as a treatment for neurocognitive diseases and disorders is expensive, requires a clinical setting, is hard to transport, is complex to use, requires external coolers, and may have severe adverse effects such as seizures. These disadvantages are the consequence of current TMS technology, which uses frequencies from 1 to 50 Hz and intense magnetic fields of around 10 million milligauss (i.e., around 1 Tesla).

There is a need for devices and methods that employ a non-invasive brain stimulation, e.g. rTMS, without using large magnetic fields, and without focusing the magnetic waves at a certain point in the brain. There is a further need for a user-friendly device that is portable, appropriate for home use, and is safe to use without the need of a medical professional to apply it.

### SUMMARY

Accordingly, the present disclosure provides low intensity and high frequency a non-invasive brain stimulation, e.g. repetitive transcranial magnetic stimulation (rTMS), methods to safely and effectively treat or prevent Alzheimer’s disease in a convenient manner.

In one aspect, the present disclosure provides a non-invasive brain stimulation, e.g. rTMS, method for treating or preventing Alzheimer’s disease, comprising repetitively applying a magnetic pulse to the scalp of a patient in need thereof thereby stimulating neurons in the brain of the patient, wherein the magnetic pulse is applied (a) repetitively over the patient’s brain (e.g., without limitation, the patient’s left prefrontal dorsolateral cortex); and (b) at a frequency of about 100 to about 150 Hz and an intensity of about 5,000 to about 15,000 milligauss (about 0.0005 Tesla to about 0.0015 Tesla).

In another aspect, the present disclosure contemplates a non-invasive brain stimulation, e.g. rTMS, method for slowing or preventing a conversion of mild cognitive impairment (MCI) to Alzheimer’s disease and/or dementia, comprising repetitively applying a magnetic pulse to the scalp of a patient in need thereof thereby stimulating neurons in the brain of the patient, wherein the magnetic pulse is applied (a) repetitively over the patient’s brain (e.g., without limitation, the patient’s left prefrontal dorsolateral cortex); and (b)at a frequency of about 100 to about 150 Hz and an intensity of about 5,000 to about 15,000 milligauss.

In various embodiments, the present disclosure contemplates applying a magnetic pulse using rTMS to a patient afflicted with MCI (e.g., amnestic or non-amnestic MCI) or Alzheimer’s disease (e.g., preclinical AD, mild AD, or moderate AD), or mild dementia. In various embodiments, the patient presents as having at least one biomarker indicative of AD, selected from high Aβ in cerebrospinal fluid, high Tau in cerebrospinal fluid, and the presence of the ApoE4 allele.

In various embodiments, treating or preventing Alzheimer’s disease comprises slowing memory loss or retaining or increasing memory capacity, memory function, or cognitive function in the patient in need thereof. In some embodiments, a method of slowing memory loss or retaining or increasing memory capacity, memory function, or cognitive function is provided, comprising administering rTMS described herein to a patient in need thereof.

In some embodiments, memory capacity, memory function, cognitive function, or memory loss is assessed using one or more of the Montreal Cognitive Assessment (MoCA), Alzheimer’s Disease Assessment Scale-Cognitive Subscale (ADAS-Cog), Verbal fluidity test, Frontal Assessment Battery (FAB), Geriatric Depression Scale (GDS-15), Clinical dementia rating (CDR), EuroQoL-5D, Daily Life Activities of Katz (ABVD), Lawton Daily Life Instrumental Activities Scale (AIVD), and Bayer Scale of Activities of Daily Living (B-ADL), Clinical Dementia Rating-Sum of Boxes (CDR-SB), and Repeatable Battery for the Assessment of Neuropsychological Status (RBANS).

In some embodiments, an increase in MoCA score or a decrease in ADAS-Cog score following administration of one or more administration events of rTMS indicates one or more of increased memory capacity, increased memory function, or increased cognitive function in the patient. In some embodiments, the treatment decreases the patient’s ADAS-COG score, as compared to the score at baseline. In some embodiments, the treatment increases the patient’s MoCA, as compared to the score at baseline.

### DESCRIPTION OF THE DRAWINGS

FIG. 1 depicts the first part of a non-limiting example of the rTMS device.

FIG. 2 depicts the second part of a non-limiting example of the rTMS device.

FIG. 3 depicts the third part of a non-limiting example of the rTMS device.

FIG. 4 depicts the instruments used for assessment of AD symptoms.

FIG. 5 depicts the comparison between baseline evaluations and the first evaluation after three months of intervention.

FIG. 6 depicts that the MCI subgroup shows a statistically different MoCA total score after 3 months of treatment. The displayed data represent averages and standard deviations.

FIG. 7 depicts that the mild dementia subgroup shows a statistically different subscore of MoCA in the Memory delayed recall with cues test. The displayed data represent averages and standard deviations.

FIG. 8 depicts the characteristics of fast gamma magnetic stimulation (FGMS). Showing in A the pattern of the magnetic field in trains, consisting of 3-second bursts of pulses at fast gamma frequency (125 Hz) alternated with 1-second without stimulation for 450 trains; while in B, they show the pulse pattern delivered.

FIG. 9 depicts the method in a diagram that goes from recruitment to analysis. The diagram explains the number of subjects in each step and the population per group

### DETAILED DESCRIPTION

In various aspects, the present disclosure provides, in part, methods for the treatment, prevention, or improvement of cognitive performance in patients (e.g., older patients) with mild cognitive impairment, Alzheimer’s disease (AD), or mild dementia using high frequency, low intensity transcranial magnetic stimulation and/or non-invasive brain stimulation using high frequency, low intensity magnetic fields.

### Repetitive Transcranial Magnetic Stimulation (rTMS)

In embodiments, the present disclosure provides methods for using non-invasive brain stimulation.

In various embodiments, the present disclosure provides for using transcranial magnetic stimulation (TMS) in a non-invasive and non-painful method to stimulate the cerebral cortex. In embodiments, the present disclosure contemplates a device that generates low-intensity pulsed magnetic field and high frequencies. In some embodiments, rTMS activates or inhibits cortical activation by generating a magnetic field that, by Faraday’s principle, generates an electrical field inside the brain. In some embodiments, the magnetic field is produced when an electric current passes through a coil. Further, in some embodiments, in order to generate an electric field inside the brain, the magnetic field starts and finishes rapidly. Without wishing to be bound by theory, if the magnetic field intensity stays constant too long, no electrical changes occur inside the brain. In addition, it is contemplated that when making use of magnetic fields of lower magnitude, neither a voltage transformer nor high currents are necessary. Indeed, the present disclosure contemplates a device that decreases potential adverse effects, facilitates its transport, and does not require external coolers for the operation.

In various embodiments, the present disclosure provides for using transcranial magnetic stimulation (TMS), wherein the magnetic pulse is applied at a frequency of about 50 to about 250 Hz, about 50 to about 200 Hz, about 100 to about 250 Hz, about 100 to about 200 Hz, about 100 to about 150 Hz, about 120 to about 135 Hz, or about 125 to about 145 Hz. In specific embodiments, the magnetic pulse is applied at a frequency of about 100 Hz, about 105 Hz, about 110 Hz, about 115 Hz, about 120 Hz, about 125 Hz, about 130 Hz, about 135 Hz, about 140 Hz, about 145 Hz, or about 150 Hz.

In embodiments, the present disclosure provides for using transcranial magnetic stimulation (TMS), wherein the magnetic pulse is applied at an intensity of about 1,000 to about 20,000 milligauss, or about 5,000 to about 15,000 milligauss, or about 7,000 to about 12,000 milligauss. In embodiments, the magnetic pulse is applied at an intensity of about 1,000 milligauss, about 2,000 milligauss, about 3,000 milligauss, about 4,000 milligauss, about 5,000 milligauss, about 6,000 milligauss, about 7,000 milligauss, about 8,000 milligauss, about 9,000 milligauss, about 10,000 milligauss, about 1,000 milligauss, about 2,000 milligauss, about 13,000 milligauss, about 4,000 milligauss, about 15,000 milligauss, about 16,000 milligauss, about 7,000 milligauss, about 18,000 milligauss, about 19,000 milligauss, or about 20,000 milligauss. In embodiments, the present disclosure provides for using transcranial magnetic stimulation (TMS), wherein the magnetic pulse is applied at an intensity of about 0.0001 to about 0.002 Tesla, or about 0.0005 to about 0.0015 Tesla, or about 0.0007 to about 0.0012 Tesla. In embodiments, the magnetic pulse is applied at an intensity of about 0.0001 Tesla, about 0.0002 Tesla, about 0.0003 Tesla, about 0.0004 Tesla, about 0.0005 Tesla, about 0.0006 Tesla, about 0.0007 Tesla, about 0.0008 Tesla, about 0.0009 Tesla, about 0.001 Tesla, about 0.0011 Tesla, about 0.0012 Tesla, about 0.0013 Tesla, about 0.0014 Tesla, about 0.0015 Tesla, about 0.0016 Tesla, about 0.0017 Tesla, about 0.0018 Tesla, about 0.0019 Tesla, or about 0.002 Tesla.

In embodiments, the present disclosure provides for using transcranial magnetic stimulation (TMS), wherein the magnetic pulse generates an electric field of about 0.1 to about 10 V/m⁷, about 0.5 to about 5 V/m⁷, or about 0.5 to about 1.5 V/m⁷. In embodiments, the magnetic pulse generates an electric field of about 1 V/m⁷.

In embodiments, the rTMS treatment method is undertaken once, or twice, or thrice, or four times daily. In particular embodiments, the method is undertaken once, twice, or thrice daily. In embodiments, the rTMS treatment method is undertaken for greater than about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the method is undertaken for about 15 to about 60 minutes. In particular embodiments, the method is undertaken for about 30 minutes. In various embodiments, the rTMS treatment method comprises magnetic pulses that are applied about 200 to about 1000 times, or about 200 to about 900 times, or about 200 to about 800 times, or about 200 to about 700 times, or about 200 to about 600 times, or about 200 to about 500 times, or about 200 to about 400 times, or about 200 to about 300 times, or about 300 to about 1000 times, or about 300 to about 900 times, or about 300 to about 800 times, or about 300 to about 700 times, or about 300 to about 600 times, or about 300 to about 500 times, or about 300 to about 400 times. In various embodiments, the rTMS treatment method comprises magnetic pulses that are applied about 300 to about 400 times. In certain embodiments, the pulses are applied about 300 times, about 310 times, about 320 times, about 330 times, about 340 times, about 350 times, about 360 times, about 370 times, about 380 times, about 390 times, or about 400 times. In various embodiments, the pulses last for about two seconds, about three seconds, about four seconds, or about five seconds. In certain embodiments, the pulses last for about four seconds, followed by one second without pulsing. In embodiments, a pulse is applied for a period of four seconds, followed by a one second pause in which no pulse is applied.

In various embodiments, the rTMS treatment method contemplated by the present disclosure is applied chronically. For example, in some embodiments, the treatment is applied for greater than about 1 month, about 2 months, about 3 months, about 4 months, about 5 months, about 6 months, about 7 months, about 8 months, about 9 months, about 10 months, about 11 months, or about 1 year, or about 2 years, or about 3 years, or about 5 years, or about 10 years. In some embodiments, the treatment is applied for the life of the patient. In some embodiments, the treatment is self-applied.

In embodiments, the rTMS treatment method contemplated by the present disclosure is applied to the patient until symptoms, such as those described herein, improve or diminish. For example, the patient may undergo the treatment and experience a recovery of cognitive ability and hope to terminate the treatment. The patient may re-start the treatment if cognitive decline reemerges. Alternatively, the patient may undergo the treatment and experience a slowing or halting of cognitive loss, but not an improvement. In such a situation, in embodiments, the patient continues treatment chronically to mitigate or prevent further cognitive loss.

### rTMS Device

In some embodiments, the present disclosure provides devices and equipment for TMS, comprising an electromagnetic field generator and a coil that emits said electromagnetic field. In further embodiments, the magnetic field is produced when an electric current passes through a coil. In some embodiments, pulses of TMS are applied repeatedly with a frequency and a determined magnitude over a cortical area in order to modulate the cerebral cortex activity in long term. In such embodiments, the method is rTMS.

It will be appreciated that the methods of the present disclosure are applied via a device capable of administering the transcranial magnetic stimulation. Indeed, in some embodiments, the TMS is applied using a device, that is suitable for conducting electric current through a coil, thus generating a magnetic field. In embodiments, the device is suitable for home use and is optionally portable. In some embodiments, the device comprises a touch screen and optionally includes “digital health” applications and tele-health solutions.

In a non-limiting example, the present disclosure contemplates a device that generates a low intensity pulsed magnetic field and variable frequencies. In some embodiments, the device is a transcranial magnetic stimulation system able to generate pulsed magnetic fields in a frequency range from 50 to 200 Hz. In further embodiments, the device is a transcranial magnetic stimulation system that generates a magnetic field with an intensity from 5,000 to 20,000 milligauss.

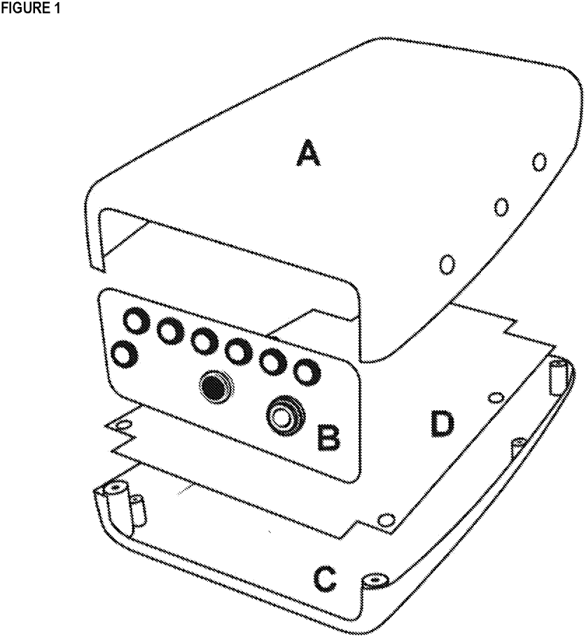

In an additional non-limiting embodiment, the TMS-generating device comprises three parts. In an embodiment, the first part of the device comprises an electronic unit that is a is a plastic box with another three parts (A), (B), (C) in the center of the box it is the electronic board (D), as depicted by FIG. 1. In embodiments, the box has six faces which are described below. In certain embodiments, the top face, with the number of part (B), is the center switch, a series of light-emitting diodes (LED) and a connector where to plug the coil cord which transmits the electromagnetic waves. The lower face is smooth, and the right face comprises three holes for ventilation. In embodiments, the left face comprises a connector to plug the power adapter cable and three holes for ventilation. In the front face, the company logo is printed and the rest is smooth. In some embodiments, the back face comprises a plastic overhang in order to fix the device to patient clothes. The electronics inside of the box consist of a PCB (Print Card Board), in which all the electronic components responsible to provide the electromagnetic waves to the coil by the connector are soldered. In embodiments, a light-emitting diode (LED) green is turned on to show the device is connected to the power adapter (12 V / 1 Amp external power supply connected to a 110/220 V socket). In an embodiment, a series of seven light-emitting diodes (LEDs) are horizontally placed in the part (B) (cover); these LEDs turn on serially to show that the device is working. In embodiments, a switch ON/OFF allows the user to start a treatment session. In embodiments, the electronic circuit is composed of three parts: (1) a pulse generator from 50 to 200 Hz, (2) an interruption generator, and (3) control of light-emitting diodes (LEDs). The electric current passes 50 to 200 times per second through the coil and the flux is interrupted every 4 seconds for 1 second. In embodiments, the circuit is able to generate a pulsed magnetic field with an approximate intensity of 5,000 to 20,000 milligauss. In embodiments, the board has transistors, resistors, heat sinks, electrolytic capacitors, and diodes in order generate the pulse to the given frequency.

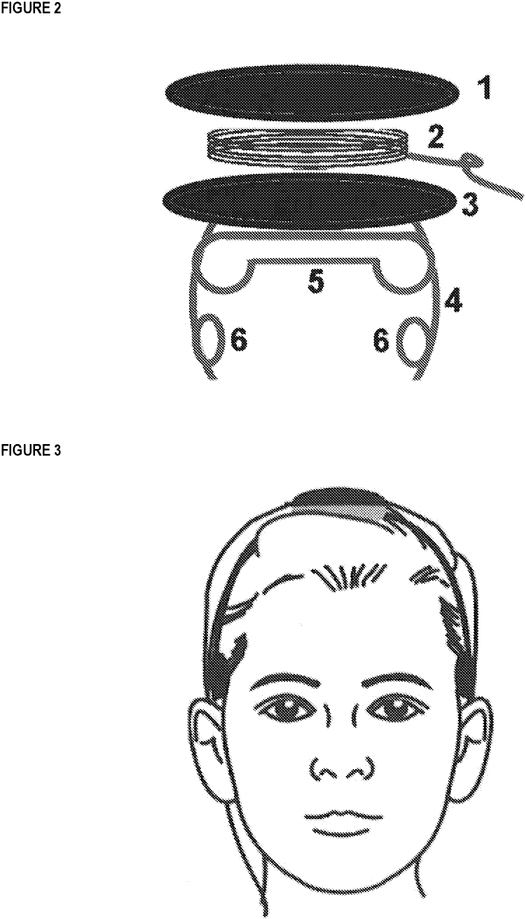

In a non-limiting embodiment, the second part of the device is made of another six subparts, as depicted in FIG. 2. In certain embodiments, the device set includes a coil that transmits the magnetic pulse as well the apparatus used to fix the device onto the patient’s head. In some embodiments, the device comprises a coil of 5 centimeters in diameter built from 50 turns of copper wire 99.5% pure, gauge 26 AWG. In further embodiments, a double coverage of insulating material covers the coil winding. In embodiments, a headband of steel of 12 centimeters in diameter (fit for any head) is fixed in the inferior part of the coil. The function of the headband is to transmit magnetic waves throughout the brain volume without using a bigger coil; without this element, the electromagnetic waves will only reach a short range in the brain. In embodiments, a rubber that adapts above the head of the patient allows a soft contact with the head of the patient. In embodiments, two movable suction cups adapt beside the head.

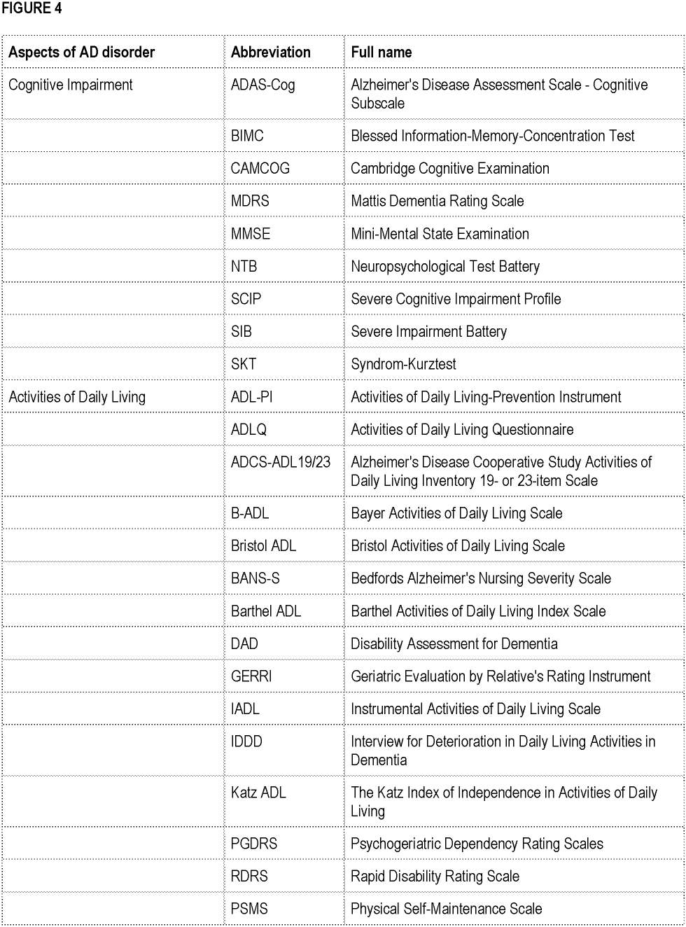

Finally, in a non-limiting embodiment, the third part of the device comprises a power adapter, as depicted in FIG. 3. In certain embodiments, the power adapter provides an alternate current to direct current transformer, and its input is from 120 to 240 Volts with a frequency of 60 Hz, and its output is from 12 V and 2 Amperes.

### Methods of Treatment

In certain aspects, the present disclosure contemplates methods for slowing or preventing a conversion of MCI to Alzheimer’s disease. In another aspect, the present disclosure contemplates methods for treating or preventing Alzheimer’s disease using non-invasive brain stimulation, e.g. repetitive transcranial magnetic stimulation (rTMS). In various embodiments, the method of rTMS comprises repetitively applying a magnetic pulse to the scalp of a patient in need thereof, thereby stimulating neurons in the brain of the patient, wherein the magnetic pulse is applied (a) repetitively over the patient’s brain (e.g., without limitation, the patient’s left prefrontal dorsolateral cortex); and (b) at a frequency of about 100 to about 150 Hz and an intensity of about 5,000 to about 15,000 milligauss. In various embodiments, methods of the present disclosure are suitable for home use and can be performed without the need for a medical professional.

In various embodiments, rTMS methods of the present disclosure stimulate neurons around about 1 to about 4 cm from the skull, about 2 to about 4 cm from the skull, about 1 to about 3 cm from the skull, or about 2 to about 3 cm from the skull. In some embodiments, the method stimulates neurons throughout the patient’s brain. In further embodiments, the method substantially stimulates neurons in the left hemisphere of the patient’s brain. In still further embodiments, the method substantially stimulates neurons in the frontal lobe of the patient’s brain. In embodiments, the method substantially stimulates neurons in the cerebral cortex of the patient. In embodiments, the method stimulates neurons outside of the patient’s left prefrontal dorsolateral cortex.

In various embodiments, the present disclosure provides for rTMS treatment methods that cause cognitive improvement but do not affect amyloid plaques and neurofibrillary tangles that may be present in the patient’s brain. That is, in embodiments, the present methods are effective in the absence of amyloid-mediated effects.

In various embodiments, the present disclosure provides for rTMS treatment methods that prevent or delay the progression of MCI to Alzheimer’s disease. In some embodiments, the treatment method improves and/or prevents diminution of cognitive traits of the patient. For example, methods of the present disclosure provide for slowing memory loss or retaining or increasing memory capacity, memory function, or cognitive function in the individual having MCI or AD. Further embodiments of the present disclosure include methods for treating psychiatric symptoms linked to MCI and Alzheimer’s disease, including, but not limited to, depression, anxiety, and sleep troubles.

### Patient Selection

In various embodiments, the present disclosure provides treatment of a patient that is afflicted with MCI, including amnestic type MCI and non-amnestic type MCI.

In various embodiments, the present disclosure provides treatment of a patient that is afflicted with preclinical Alzheimer’s disease, mild Alzheimer’s disease, or moderate Alzheimer’s disease. In further embodiments, the patient is afflicted with mild dementia.

In some embodiments, the patient afflicted with MCI or Alzheimer’s disease and/or dementia presents as having at least one biomarker indicative of AD, selected from high Aβ in cerebrospinal fluid, high Tau in cerebrospinal fluid, and the presence of the ApoE4 allele.

In further embodiments, the patient presents as having a test score of one or more identified instruments for assessment of AD symptoms that is considered to indicate the presence of cognitive impairment or AD. For example, in embodiments, the patient presents as having a MoCA test score of less than about 26; an ADAS-Cog test score of 2, 3, 4, or 5 on one or more subscores of the test; or a CDR score of at least about 0.5, at least about or 1, or at least about 2.

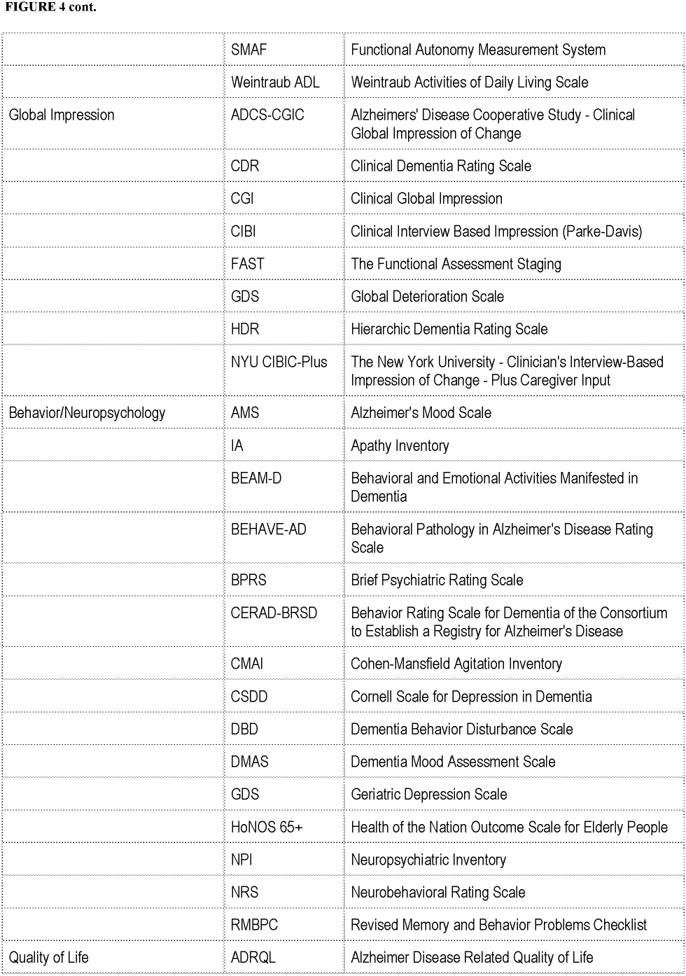

In further embodiments, the patient presents as having a test score of one or more identified instruments for assessment of AD symptoms that is considered to indicate the presence of cognitive impairment or AD, such tests being found in FIG. 4 herein.

### Mild Cognitive Impairment (MCI)

In some embodiments, the present disclosure provides methods for slowing or preventing a conversion of MCI to Alzheimer’s disease and/or dementia. Mild cognitive impairment causes cognitive changes that are serious enough to be noticed to the person affected and to family members and friends, but do not affect the individual’s ability to carry out everyday activities. Approximately 15% to 20% of people age 65 or older have MCI. In embodiments, patients presenting with MCI, especially MCI involving memory problems, eventually develop Alzheimer’s disease or other dementias.

In various embodiments, the MCI is amnestic MCI, which primarily affects memory. In embodiments, a person having amnestic MCI may start to forget important information that he or she would previously have recalled easily, such as appointments, conversations or recent events. In further embodiments, the MCI is non-amnestic MCI, which affects thinking skills other than memory, including the ability to make sound decisions, judge the time or sequence of steps needed to complete a complex task, or visual perception.

In embodiments, risk factors strongly linked to MCI include: advancing age, family history of Alzheimer’s or another dementia, and conditions that raise risk for cardiovascular disease.

In some embodiments, patients practice forms of prevention against or mitigation of MCI, including, but not limited to, controlling their blood sugar (e.g., lowering systolic blood pressure), exercising on a regular basis, avoiding smoking and heavy drinking, losing excess weight, limiting caffeine, reducing stress, and eating a diet low in fat, cholesterol, and sodium.

### Alzheimer’s Disease (AD)

In various embodiments, the present disclosure provides methods of treating or preventing Alzheimer’s disease (AD). Specifically, rTMS can be administered to treat Alzheimer’s disease (AD), which is a progressive, neurodegenerative disease characterized in the brain by abnormal clumps (amyloid plaques) and tangled bundles of fibers (neurofibrillary tangles) composed of misplaced proteins. Repetitive transcranial magnetic stimulation can be used to delay onset, reduce severity, and reduce the rate of progression of Alzheimer’s disease. In addition, rTMS can be used to treat long-term survivors as well as patients who exhibit more rapid disease progression. The severity and progression of, and improvement in, Alzheimer’s disease can be assessed by a number of criteria, including Activities of Daily Living scales, one of which is the Progressive Deterioration Scale (PDS), in which 29 questions are used to determine a patient’s ability to perform functions such as interacting socially, travelling without getting lost, coping when the caregiver is away, and identifying or using common household objects such as telephones; the Mini-Mental State Examination (MMSE), which is a short collection of cognitive tests that examines several areas of cognition and is easy to administer and score and is widely used to measure the onset, progression and severity of Alzheimer’s disease.

Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. In some implementations, subjects who are the progeny of an AD patient or of a carrier of a mutation in a gene tied to AD, or who are identified as having Alzheimer’s disease (e.g., by a genetic test), are given a prophylactic dose of rTMS, e.g., prior to showing physical symptoms of AD.

Symptoms of AD include memory loss, language deterioration, impaired ability to mentally manipulate visual information, poor judgment, confusion, restlessness, and mood swings. Eventually AD destroys cognition, personality, and the ability to function. In some embodiments, patients suffering from Alzheimer’s disease experience psychiatric symptoms, including, but not limited to, depression, anxiety, and sleep troubles.

In some embodiments, early Alzheimer’s disease (early AD) includes patients with mild cognitive impairment, such as a memory deficit, due to AD and patients having AD biomarkers, for example amyloid positive patients, patients having a positive florbetapir PET scan, or patients having a positive Tau PET scan. The early symptoms of AD, which include forgetfulness and loss of concentration, are often missed because they resemble natural signs of aging. AD is a progressive disease, but its course can vary from 5 to 20 years. rTMS can be used to ameliorate one or more these symptoms, or to reduce the rate of disease progression.

Treatments for AD include donepezil (ARICEPT), rivastigmine (EXELON), and galantamine (RAZADYNE), memantine (NAMENDA), and other drugs that may affect AD progression include nonsteroidal anti-inflammatory drugs (NSAIDS), statins, folic acid, gingko biloba, and vitamins E, B6, and B12. Any one or more of these treatments can be used in combination with rTMS.

The present disclosure contemplates in various embodiments that use of rTMS as a combination therapy with a treatment for AD (e.g., donepezil (ARICEPT), rivastigmine (EXELON), and galantamine (RAZADYNE), memantine (NAMENDA)) reduces side effects associated with AD pharmaceutical drugs. In some embodiments, use of rTMS as a combination therapy with a treatment for AD reduces the frequency and dosage of use of AD pharmaceutical drugs.

### Dementia

Cognition in the elderly extends across a spectrum that goes from the normal aging process to dementia. Dementia is a severe decline of cognitive function that affects the daily life function of the subject. This decline has different etiologies among which include Alzheimer’s disease, vascular damage, frontotemporal lobar degeneration and Lewy body dementia.

Although the main risk factor to develop dementia is age, this is not an inevitable consequence of aging. In some embodiments, a patient presents with one or more of the presence of APOE ε4 allele, history of traumatic brain injury, depression, heart disease, diabetes mellitus, hypertension, obesity and tobacco use. There are treatments that can improve neurodegenerative disorders related to dementia. All existing treatments are directed to improve neurotransmission through inhibitors of acetylcholinesterase (donepezil, rivastigmine, galantamine) and inhibitors of NMDA receptors, such as memantine. Although these molecules can have beneficial effects in the stabilization of some cognitive symptoms, they do not modify the prognosis of the disease. Any one or more of existing dementia treatments can be used in combination with rTMS.

### Monitoring/Assessing Response to rTMS Treatment

In some embodiments, a patient being treated with rTMS described herein is monitored or assessed to determine if the patient is benefiting from the treatment. In some embodiments, a therapeutic benefit is a slowing, delay, or cessation of the progression of AD, or a reduction in clinical, functional, or cognitive decline. For example, benefits may include, but are not limited to, (1) inhibition, to some extent, of disease progression, including slowing of progression and complete arrest; (2) improvement in one or more assessment metrics, including but not limited to ADAS-Cog, MoCA, Verbal fluidity, FAB, GDS-15, CDR, EuroQoL-5D, ABVD, AIVD, B-ADL, CDR-SB, and RBANS, and one or more of the instruments listed in FIG. 4; 3) improvement in daily functioning and/or psychiatric symptoms (e.g., depression, anxiety, and/or sleep troubles) of the patient; (4) a decrease in a biomarker indicative of AD, selected from high Aβ in cerebrospinal fluid and high Tau in cerebrospinal fluid; and (5) an increase in a biomarker indicative of improvement of AD. Patients may be assessed using any measure that can detect a benefit to the patient.

In some embodiments, the cognitive ability and/or daily functioning of the patient is assessed prior to, during, and/or after a course of administration of rTMS described herein. Various cognitive and functional assessment tools are known in the art, and may be used to assess, diagnose, and/or score mental function, cognition, and/or neurological deficit. Illustrative tools include, but are not limited to, MoCA, ADAS-Cog (including ADAS-Cog 12, ADAS-Cog 13, and ADAS-Cog 14), CDR, CDR-SB, Verbal fluidity, FAB, GDS-15, EuroQoL-5D, ABVD, AIVD, and B-ADL. Additional instruments used for assessment of AD symptoms are known in the art and provided in FIG. 4.

In various embodiments, one or more of In some embodiments, a patient that has been treated with rTMS described herein shows an improvement (i.e., an increase or a decrease) in the patient’s score of one or more instruments described in FIG. 4 as compared to baseline (e.g., prior to treatment or at an earlier timepoint during treatment). In some embodiments, the patient’s score of one or more instruments described in FIG. 4 increases or decreases by at least 5%, or by at least 10%, or by at least 15%, or by at least 20%, or by at least 25%, or by at least 30%, or by at least 35%, or by at least 40%, or by at least 45%, or by at least 50%, or by at least 55%, or by at least 60%, or by at least 65%, or by at least 70%, or by at least 75%, or by at least 80%, or by at least 85%, or by at least 90%, or by at least 95%. In some embodiments, a patient that has been treated with rTMS described herein shows a slowing of the rate of increase or decrease in their score of one or more instruments described in FIG. 4 by at least 5%, or by at least 10%, or by at least 15%, or by at least 20%, or by at least 25%, or by at least 30%, or by at least 35%, or by at least 40%, or by at least 45%, or by at least 50%, or by at least 55%, or by at least 60%, or by at least 65%, or by at least 70%, or by at least 75%, or by at least 80%, or by at least 85%, or by at least 90%, or by at least 95%. In some embodiments, a stable score of one or more instruments described in Table compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In some embodiments, the Montreal Cognitive Assessment (MoCA) Test is a rapid screening instrument for mild cognitive dysfunction, including MCI. In further embodiments, the MoCA, in a 30-point format, assesses: short term memory; visuospatial abilities; executive functions; attention; concentration and working memory, language; and orientation to time and place. MoCA scores range between 0 and 30. In various embodiments, a score of 26 or over is considered to be normal. In an illustrative study, people without cognitive impairment scored an average of 27.4; people with MCI scored an average of 22.1; people with Alzheimer’s disease scored an average of 16.2. The short-term memory recall task (5 points) involves two learning trials of five nouns and delayed recall after approximately five minutes. Visuospatial abilities are assessed using a clock-drawing task (3 points) and a three-dimensional cube copy (1 point). Multiple aspects of executive functions are assessed using an alternation task adapted from the trail-making B task (1 point), a phonemic fluency task (1 point), and a two-item verbal abstraction task (2 points). Attention, concentration, and working memory are evaluated using a sustained attention task (target detection using tapping; 1 point), a serial subtraction task (3 points), and digits forward and backward (1 point each). Language is assessed using a three-item confrontation naming task with low-familiarity animals (3 points), repetition of two syntactically complex sentences (2 points), and the fluency task. Abstract reasoning is assessed using a “describe the similarity” task with 2 points being available. Orientation to time and place is evaluated by asking the subject for the date and the city in which the test is occurring (6 points).

In some embodiments, a patient that has been treated with rTMS described herein shows an improvement (i.e., an increase) in the patient’s MoCA score compared to baseline (e.g., prior to treatment or at an earlier timepoint during treatment). In some embodiments, the patient’s MoCA score increases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their MoCA score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable MoCA score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In some embodiments, the Alzheimer’s Disease Assessment Scale-Cognitive Subscale (ADAS-Cog) is used to assess the severity of cognitive symptoms of dementia. The ADAS-Cog is considered the gold standard for assessing the efficacy of antidementia treatments. The full ADAS takes about 45 minutes to administer, and is scored from 0 to 150 by summing the number of errors made on each task so that higher scores indicate worse performance. This instrument allows exploring the cognitive processes commonly affected in Alzheimer’s disease, including 11 items that can be scored from 0 to 5, having a maximum of 70 points, the higher the score, the greater the degree of alteration. The concurrent validity with the Dementia Rating Scale was r = 0.642 (p <0.001), the interobserver reliability was 0.650-0.989 (p <0.01) for patients with Alzheimer’s disease and 0.658-1 (p <0.01) for cognitively normal, the test-retest reliability was 0.579 (p <0.05) -0.919 (p <0.001). Considering an adjustment of the age and education level score, the cut-off point of the ADAS-Cog was ≥12 (sensitivity: 89-19%, specificity: 88.53%), the area under the ROC curve was 0.95, 0.94. In further embodiments, the version of the ADAS-Cog includes 13 items (which is referred to as “ADAS-Cog 13”). See, e.g., Rosen et al., 1984, Amer. J. Psych. 141: 1356-1364; Mohs et al., 1997, Alzheimer’s disease Assoc. Disorders, 11(2): S13-S21. The ADAS-Cog 13 is a multi-part cognitive assessment that assesses multiple cognitive domains, including memory, naming, word finding, comprehension, praxis, attention, orientation, and spontaneous speech. The ADAS-Cog 13 is based on the ADAS-Cog and additionally includes delayed word recall and a number cancellation task. The ADAS-Cog 13 produces scores up to 85 points. In some embodiments, a decrease in ADAS-Cog 13 score is indicative of improvement in the patient’s condition, whereas an increase in ADAS-Cog 13 score is indicative of worsening of the patient’s condition. In some embodiments, a stable ADAS-Cog 13 score is indicative of a slowing, delay, or cessation of the progression of AD, or a reduction in clinical or cognitive decline.

In some embodiments, a patient that has been treated with rTMS described herein shows an improvement (i.e., a decrease) in the patient’s ADAS-Cog score (e.g., ADAS-Cog 13) compared to baseline (e.g., prior to treatment or at an earlier timepoint during treatment). In some embodiments, the patient’s ADAS-Cog score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their ADAS-Cog score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable ADAS-Cog score compared to baseline (e.g., prior to treatment or at an earlier timepoint during treatment) may be indicative of a slowing, delay, or cessation of the progression of AD, or a reduction in clinical or cognitive decline.

In various embodiments, the Clinical dementia rating (CDR) is calculated on the basis of testing six different cognitive and behavioral domains such as memory, orientation, judgment and problem solving, community affairs, home and hobbies performance, and personal care. In some embodiments, the CDR is based on a scale of 0-3: no dementia (CDR = 0), questionable dementia (CDR = 0.5), MCI (CDR = 1), moderate cognitive impairment (CDR = 2), and severe cognitive impairment (CDR = 3).

In some embodiments, a patient that has been treated with rTMS described herein shows an improvement (i.e., a decrease) in the patient’s CDR score compared to baseline (e.g., prior to treatment or at an earlier timepoint during treatment). In some embodiments, the patient’s CDR score decreases from 3 to 2, or from 3 to 1, or from 3 to 0.5, or from 3 to 0, or from 2 to 1, or from 2 to 0.5, or from 2 to 0, or from 1 to 0.5, or from 1 to 0, or from 0.5 to 0. In some embodiments, a patient that has been treated with rTMS described herein shows a slowing of the rate of increase in their CDR score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable CDR score compared to baseline (e.g., prior to treatment or at an earlier timepoint during treatment) may be indicative of a slowing, delay, or cessation of the progression of AD, or a reduction in clinical or cognitive decline.

In various embodiments, the Frontal Assessment Battery (FAB) is a screening test that allows the investigator to explore executive functioning through 6 items that include: go/no-go tasks, interference control, verbal fluency, and motor programming. In some embodiments, the FAB explores conceptualization, mental flexibility, motor programming, sensitivity to interference, inhibitory control, and environmental autonomy. A cut-off point of ≤11 has been established as an indication of alterations in these cognitive functions.

In some embodiments, a patient that has been treated with rTMS described herein shows an improvement (i.e., an increase) in the patient’s FAB score compared to baseline (e.g., prior to treatment or at an earlier timepoint during treatment). In some embodiments, the patient’s FAB score increases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their FAB score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable FAB score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

The verbal fluency test (Duff et al., 2004 Neurology. 2004;62:556-562; Ostrosky & Ardila, 1999 J. Int′l. Neuropsyc. Soc′y., 5: 413-433) incorporates a task that asks the patient to generate as many words as quickly as possible according to two criteria: semantic (animals) and phonological (letter F). The cut-off points of this task are determined by age and education level.

In some embodiments, a patient that has been treated with rTMS described herein shows an improvement (i.e., an increase) in the patient’s verbal fluency test score compared to baseline (e.g., prior to treatment or at an earlier timepoint during treatment). In some embodiments, the patient’s verbal fluency test score increases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their verbal fluency test score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable verbal fluency test score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

The Index of Independence in Daily Life Activities of Katz (ABVD) evaluates the degree of functional independence in six basic activities (each worth 1 point): bath, clothing, use of the toilet, mobility, continence, and food. A functional dependency is considered in basic activities when at least one of these activities is lost. A score of 6 points indicates high independence, while a score of 0 indicates high dependence.

In some embodiments, a patient that has been treated with rTMS described herein shows an improvement (i.e., an increase) in the patient’s ABVD score compared to baseline (e.g., prior to treatment or at an earlier timepoint during treatment). In some embodiments, the patient’s ABVD score increases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their ABVD score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable ABVD score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In various embodiments, the Lawton Daily Life Instrumental Activities Scale (AIVD) rates 8 items with a value of 1 (independent) and 0 (dependent). The final score is the sum of the value of all the answers, it can be between 0 (total dependence) and 8 (total independence), however, an adjustment is suggested to reduce gender bias, eliminating three activities for men: kitchen, home care, and laundry, resulting in a total of 5 points. Functional independence is considered when all activities are preserved.

In some embodiments, a patient that has been treated with rTMS described herein shows an improvement (i.e., an increase) in the patient’s AIVD score compared to baseline (e.g., prior to treatment or at an earlier timepoint during treatment). In some embodiments, the patient’s AIVD score increases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their AIVD score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable AIVD score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In some embodiments, the Bayer Scale for Daily Life Activities (B-ADL) is an instrument applied to the informant for the early detection of dementia, and it consists of 25 items, of which 5 measure basic or low cognitive demand activities and the rest more complex activities. The rating is determined either by the total score or by the index calculated from the total score among the applicable items. Its specificity and sensitivity values are 0.72 and 0.81 respectively for the detection of dementia, values for mild cognitive impairment are not yet described. A global score for the B-ADL scale is computed by summating across all items without the inclusion of those rated as “unknown” or “not applicable.” The total is then divided by the number of items rated “1” to “10.” The resulting score ranges between 1 and 10. Two subscores were computed; items 5, 7, and 19 are everyday tasks requiring short- and long-term memory (subscore 1) and items 21 to 25 relating to cognitive functions important for carrying out everyday life activities (subscore 2). The scores are summed and then divided by the number of relevant items rated “1” to “10.” Higher score corresponds to more severe deficits.

In some embodiments, a patient that has been treated with rTMS described herein shows an improvement (i.e., a decrease) in the patient’s B-ADL score compared to baseline (e.g., prior to treatment or at an earlier timepoint during treatment). In some embodiments, the patient’s B-ADL score decreases from 3 to 2, or from 3 to 1, or from 3 to 0.5, or from 3 to 0, or from 2 to 1, or from 2 to 0.5, or from 2 to 0, or from 1 to 0.5, or from 1 to 0, or from 0.5 to 0. In some embodiments, a patient that has been treated with rTMS described herein shows a slowing of the rate of increase in their B-ADL score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable B-ADL score compared to baseline (e.g., prior to treatment or at an earlier timepoint during treatment) may be indicative of a slowing, delay, or cessation of the progression of AD, or a reduction in clinical or cognitive decline.

In various embodiments, the Abbreviated geriatric depression scale (GDS-15) consists of 15 questions whose answers are dichotomous. A score greater than or equal to 6 points indicates depression. Internal consistency values of 0.94, test-retest reliability of 0.85 and halving reliability of 0.94 are reported. Convergent validity values of 0.83 have been reported with the Hamilton Depression Assessment Scale and high discrimination in older adults.

In some embodiments, a patient that has been treated with rTMS described herein shows an improvement (i.e., a decrease) in the patient’s GDS-15 score compared to baseline (e.g., prior to treatment or at an earlier timepoint during treatment). In some embodiments, the patient’s GDS-15 score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their GDS-15 score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable GDS-15 score compared to baseline (e.g., prior to treatment or at an earlier timepoint during treatment) may be indicative of a slowing, delay, or cessation of the progression of AD, or a reduction in clinical or cognitive decline.

In various embodiments, the EuroQol 5-D (EQ-5D) is a questionnaire composed of three parts that allow exploring the quality of life of patients. The first part allows the respondent to define the state of health through five dimensions: mobility, personal care, daily activities, pain/discomfort, and anxiety/depression. Each dimension has three levels of severity (no problems, some problems or moderate problems and more serious problems). The combination of the values of all dimensions generates a 5-digit code that determines the patient’s health profile. The second part is a visual scale graduated from 0 to 100, where the greater the better state of health perceived. The third part collects other data that allow the demographic characterization of the studied group. This scale has shown satisfactory convergent validity compared to other measures of quality of life, as well as adequate discriminant validity. The test-retest reliability is, overall, moderate to good.

In some embodiments, a patient that has been treated with rTMS described herein shows an improvement (i.e., a decrease) in the patient’s EQ-5D five-digit score compared to baseline (e.g., prior to treatment or at an earlier timepoint during treatment). In some embodiments, the patient’s EQ-5D five-digit score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their EQ-5D five-digit score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable EQ-5D five-digit score compared to baseline (e.g., prior to treatment or at an earlier timepoint during treatment) may be indicative of a slowing, delay, or cessation of the progression of AD, or a reduction in clinical or cognitive decline.

In some embodiments, the Mini-Mental State Examination (MMSE) provides a 30-point questionnaire that measures cognitive impairment. In embodiments, any MMSE score of 24 or more (out of 30) indicates a normal cognition. Below this, scores indicate severe (≤9 points), moderate (10-18 points) or mild (19-23 points) cognitive impairment. The raw score may also need to be corrected for educational attainment and age.

In some embodiments, a patient that has been treated with rTMS described herein shows an improvement (i.e., an increase) in the patient’s MMSE score compared to baseline (e.g., prior to treatment or at an earlier timepoint during treatment). In some embodiments, the patient’s MMSE score increases by at least 5%, or by at least 10%, or by at least 15%, or by at 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their MMSE score by at least 5%, or by at least 10%, or by at least 15%, at least 20%, or by at least 25%, or by at least 30%, or by at least 35%, or by at least 40%, or by at least 45%, or by at least 50%. In some embodiments, a stable MMSE score compared to baseline (e.g., prior to treatment or at an earlier timepoint during treatment) may be indicative of a slowing, delay, or cessation of the progression of AD, or a reduction in clinical or cognitive decline.

In some embodiments, the Clinical Dementia Rating-Sum of Boxes (CDR-SB) provides a score between 0 and 18. See, e.g. O′Bryant et al., 2008, Arch Neurol 65: 1091-1095. CDR-SB score is based on semi-structured interviews of patients and caregiver informants, and yields five degrees of impairment in performance for each of six categories of cognitively-based functioning: memory, orientation, judgment and problem solving, community affairs, home and hobbies, and personal care. Each category is scored from 0-3 (the five degrees are 0, 0.5, 1, 2, and 3). The sum of the score for the six categories is the CDR-SB score. In various embodiments, a decrease in CDR-SB score is indicative of improvement in the patient’s condition, whereas an increase in CDR-SB score is indicative of worsening of the patient’s condition. In some embodiments, a stable CDR-SB score is indicative of a slowing, delay, or cessation of the progression of AD, or a lack of appearance of new clinical, functional, or cognitive symptoms or impairments, or an overall stabilization of disease activity.

In some embodiments, a patient that has been treated with rTMS described herein shows an improvement (i.e., a decrease) in the patient’s CDR-SB score compared to baseline (e.g., prior to treatment or at an earlier timepoint during treatment). In some embodiments, the patient’s CDR-SB score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their CDR-SB score by at least 15%, or by at least 20%, or by at least 25%, or by at least 30%, or by at least 35%, or by at least 40%, or by at least 45%, or by at least 50%. In some embodiments, a stable CDR-SB score compared to baseline (e.g., prior to treatment or at an earlier timepoint during treatment) may be indicative of a slowing, delay, or cessation of the progression of AD, or a lack of appearance of new clinical, functional, or cognitive symptoms or impairments, or an overall stabilization of disease activity.

In further embodiments, Repeatable Battery for the Assessment of Neuropsychological Status (RBANS), consists of twelve subtests that are combined to provide five indices, one for each of the five domains tested (immediate memory, visuospatial/constructional, language, attention, and delayed memory). See, e.g., Randolph et al., 1998, J Clin Exp Neuropsychol 20: 310-319. Extensive normative values are provided in the testing manuals. In some embodiments, an increase in RBANS score is indicative of improvement in the patient’s condition, whereas a decrease in RBANS score is indicative of worsening of the patient’s condition. In some embodiments, a stable RBANS score is indicative of a slowing, delay, or cessation of the progression of AD, or a reduction in clinical or cognitive decline.

In some embodiments, a patient that has been treated with rTMS described herein shows an improvement (i.e., an increase) in the patient’s RBANS score compared to baseline (e.g., prior to treatment or at an earlier timepoint during treatment). In some embodiments, the patient’s RBANS score increases by at least 5%, or by at least 10%, or by at least 15%, or by at 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their RBANS score by at least 5%, or by at least 10%, or by at least 15%, at least 20%, or by at least 25%, or by at least 30%, or by at least 35%, or by at least 40%, or by at least 45%, or by at least 50%. In some embodiments, a stable RBANS score compared to baseline (e.g., prior to treatment or at an earlier timepoint during treatment) may be indicative of a slowing, delay, or cessation of the progression of AD, or a reduction in clinical or cognitive decline.

### Additional Therapeutic Agents and Combination Therapy

Administration of rTMS may be combined with additional therapeutic agents. Co-administration of the additional therapeutic agent and the present rTMS may be simultaneous or sequential.

In one embodiment, the additional therapeutic agent and the rTMS are administered to a subject simultaneously. The term “simultaneously” as used herein, means that the additional therapeutic agent and the rTMS are administered with a time separation of no more than about 60 minutes, such as no more than about 30 minutes, no more than about 20 minutes, no more than about 10 minutes, no more than about 5 minutes, or no more than about 1 minute.

In a further embodiment, the additional therapeutic agent and the rTMS are administered to a subject simultaneously but the release of the additional therapeutic agent from its respective dosage form and the rTMS may occur sequentially.

Co-administration does not require the additional therapeutic agent and the rTMS to be administered simultaneously, if the timing of their administration is such that the pharmacological activities of the additional therapeutic agent and the administration of rTMS overlap in time. For example, the additional therapeutic agent and the rTMS can be administered sequentially. The term “sequentially” as used herein means that the additional therapeutic agent and the rTMS are administered with a time separation of more than about 60 minutes. For example, the time between the sequential administration of the additional therapeutic agent and the administration of rTMS can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent being administered. Either the additional therapeutic agent or the rTMS may be administered first.

In various embodiments, the present disclosure contemplates rTMS methods used in tandem with one or more additional therapeutic agents. In certain embodiments, the rTMS methods of the present disclosure obviate the need for treatment with one or more additional therapeutic agents. In some embodiments, the additional therapeutic agent is selected from neurological drugs, corticosteroids, antibiotics, antiviral agents, anti-Tau antibodies, Tau inhibitors, anti-amyloid beta antibodies, beta-amyloid aggregation inhibitors, anti-BACE1 antibodies, and BACE1 inhibitors. In specific embodiments, the additional therapeutic agent is a treatment for AD, including, but not limited to, donepezil (ARICEPT), rivastigmine (EXELON), galantamine (RAZADYNE), and memantine (NAMENDA). In various embodiments, the rTMS treatment of the present disclosure is substantially free of adverse effects, optionally selected from epileptic seizures, nausea, and headache.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of a stimulus, relative to the absence of such stimulus.

As used herein, the word “include,” and its variants, is intended to be non-limiting, such that recitation of items in a list is not to the exclusion of other like items that may also be useful in the treatments and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

An effective amount of the treatment as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: Clinical Study to Evaluate Improvement of Patient Cognition Using High Frequency, Low Intensity (HFLI) Transcranial Magnetic Fields (TMS) at Fast Gamma Frequencies.

The purpose of this double-blinded, sham-controlled clinical trial was to ascertain whether repetitive transcranial magnetic stimulation, as applied by an at-home device, can improve patients’ cognitive performance and/or reduce cognitive symptoms of Alzheimer’s disease (e.g., by assessment of ADAS-Cog scores).

Patients that met inclusion criteria were invited to participate as part of either the MCI group or the Mild Dementia group. For the MCI group, inclusion criteria included: patients of both sexes; over 65 years old; written informed consent; patients already in follow-up by a qualified physician or who have completed clinical and neuropsychological diagnostic evaluation compatible with mild cognitive impairment by Petersen criteria; CDR 0.5; preserved or corrected ability to see and hear; and have a caregiver. For the Mild Dementia group, inclusion criteria included: patients of both sexes; over 65 years old; written informed consent; patients already in follow-up by a qualified physician that meet the NINCDS-ADRDA criteria; CDR 1; patients may have drug treatment for dementia without change for the last three months preserved or corrected ability to see and hear; and have a caregiver.

Patients who met all inclusion criteria, understood the consent and wanted to participate in the study were recruited and included in the clinical study. Once the participant was recruited, clinical history and physical examination were performed.

Patients were then randomized in two groups: (1) the first group received sham high-frequency low-intensity transcranial magnetic stimulation (HFLI TMS); and (2) the other group received real HFLI TMS. Once the evaluation was finished, the participant was given their corresponding device (sham or real) and advice on equipment use. The patient applied the HFLI TMS device for 30 minutes per treatment, daily, every 12 hours, for 6 months. The device was used by placing a headband connected to the device (which was connected to an electric current) on the patient’s head. The device had a sensor that allows the wireless communication of the usage time directly to the manufacturer. The intervention was dichotomous—either real rTMS or placebo rTMS. The real rTMS emitted a pulsating magnetic field of 125 Hz of frequency with an intensity of 10 thousand milligauss. The placebo rTMS was applied with a physically identical device to the real rTMS; however, this device will not emit a magnetic field whatsoever.

A baseline assessment was done in all patients before starting daily HFLI TMS sessions, after 3 months of sessions and after 6 months of sessions. The following neuropsychological tests were applied during the patient’s initial evaluation: the Montreal Cognitive Assessment (MoCA), Alzheimer’s Disease Assessment Scale-Cognitive Subscale (ADAS-Cog), Verbal fluidity test, Frontal Assessment Battery (FAB), Geriatric Depression Scale (GDS-15), Clinical dementia rating (CDR), EuroQoL-5D, Daily Life Activities of Katz (ABVD), Lawton Daily Life Instrumental Activities Scale (AIVD), and Bayer Scale of Activities of Daily Living (B-ADL). These test results inform the comparative baseline scores. The following neuropsychological tests were applied during the three-month evaluation: MoCA, CDR, EuroQoL-5D, ADL, IADL. The following neuropsychological tests were applied during the six-month evaluation: ADAS-Cog, MoCA, Verbal fluidity, FAB, GDS-15, CDR, EuroQoL-5D, ABVD, AIVD, and B-ADL.

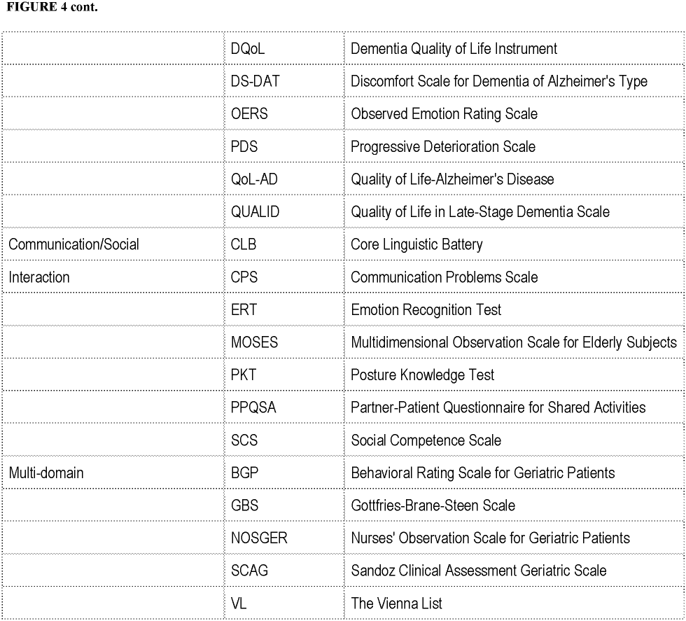

The current results of the study are depicted in FIG. 5, showing the comparison between baseline evaluations and the first evaluation after three months of intervention. The data shows positive changes in MoCA total score in the MCI population and positive changes in the Memory-delayed (cued recall) subscore of MoCA in the mild dementia population. As shown, results of 11 subjects of the clinical trial suggest that the MCI subgroup shows a statistically different MoCA total score after 3 months of treatment. The mild dementia subgroup shows a statistically different in the Memory delayed recall with cues subscore of MoCA. The displayed data represent averages and standard deviations. P values were calculated with a Wilcoxon signed-rank test.

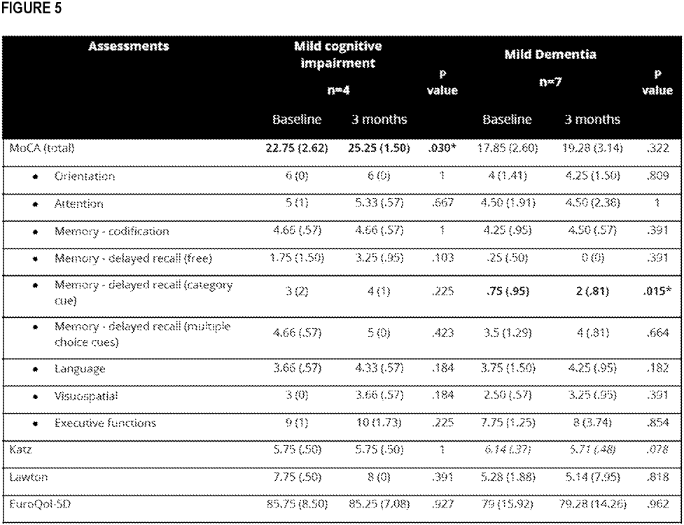

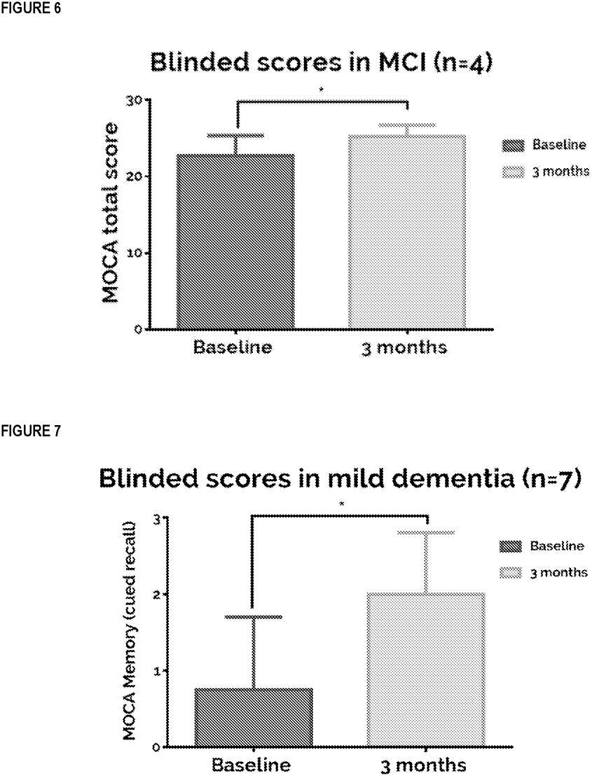

FIG. 6 depicts that the MCI subgroup shows a statistically different MoCA total score after 3 months of treatment. FIG. 7 depicts that the mild dementia subgroup shows a statistically different in the Memory delayed recall with cues subscore of MoCA. The displayed data represent averages and standard deviations.

The current clinical results show that HFLI magnetic fields at fast gamma frequencies lead to improvement in cognition.

### Example 2: A Pilot Study of the Use of Fast Gamma Magnetic Stimulation Over the Left Prefrontal Dorsolateral Cortex for the Treatment of MCI and Mild Alzheimer’s Disease

We performed a parallel randomized sham-controlled proof of concept clinical trial with a 1:1 allocation.

All participants were assessed for eligibility for the trial during their consultation in a memory clinic. For eligibility, all subjects had to be over 65 years old and had to be diagnosed with MCI or mild AD by an attending by geriatrician/neurologist and neuropsychologist the participants were classified into two groups (MCI and AD) according to their performance in neuropsychological evaluation and current clinical criteria. For the diagnosis of AD, the criteria of the Diagnostic and Statistical Manual of Mental Disorders version 5 and the criteria of the National Institute of Neurological and Communicative Disorders and Stroke - Alzheimer’s Disease and Related Disorders Association (NINCDS-ADRDA) were used. For the dementia stage, the CDR score (Mild = 1) and participants with MCI diagnosis was established according to Petersen’s criteria. They also needed to have a stable dementia medication for at least 3 months before starting any intervention. Subjects with uncontrolled medical conditions, diagnosis of major depressive disorder, metal implants, previous history of seizures and previous utilization of any brain stimulation devices were excluded from the study. After determining their eligibility, patients were invited to participate in the study and were asked to participate voluntarily and sign an informed consent.

Participants were evaluated twice, at enrollment and after the six months of intervention. In each evaluation, subjects were interrogated for any adverse effect, and completed a standardized neuropsychological assessment.

Subjects were randomized to either receive daily sessions two times a day of FGMS for 6 months or daily sessions two times a day of sham FGMS for 6 months. The device used in this study was designed and manufactured by Actipulse Neuroscience (Boston, USA); it is portable, can be used at home without medical supervision and works by passing electric current into a coil to generate a rapidly changing magnetic field at fast gamma frequency. The coil had a circular shape with a 6 cm diameter and 50 turns of copper; it was also mounted over a headband to assure that it was positioned correctly by the patient.

Subjects were instructed by a researcher on how to position the coil over the F3 coordinate of the 10-20 EEG system (left prefrontal dorsolateral cortex) and an illustrated manual with the instructions was also provided to assure that the coil was correctly positioned. Also, the device included a wireless sensor that allowed researchers to evaluate treatment compliance for each patient.

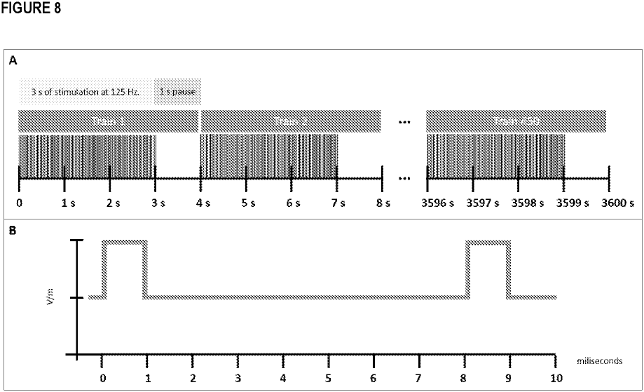

The coil of the device emitted a patterned magnetic field in trains, consisting of 3-second bursts of pulses at fast gamma frequency (125 Hz) alternated with 1-second without stimulation (see FIG. 8 for more details about the stimulation pattern); a total of 450 trains (30 minutes of stimulation) were applied in each session. Each pulse had an approximate magnetic field intensity of 0.5 milliteslas. Adherence was assessed weekly by a blinded researcher through a computerized program which registered signals emitted by the device when it was connected and turned on; subjects with adherence lower than 80% were eliminated from the study.

A block randomization was used to assign devices to perform any of the two possible interventions. Eight blocks with a fixed size of 10 were created using a computer random number generator. One researcher, who had no interaction with research subjects or other researchers, knew which device corresponded to active or sham intervention and the actual block size. The sham and the active devices were identical for the exception that one device did not emit any kind of magnetic field from the coil. As the intensity of the magnetic field is low, no sensory cues were evoked by the stimulation that could compromise blinding from the research on the subject side. Each device was labeled from 1 to 80 and were assigned consecutively to each patient entering the trial. Researchers that were involved in patient recruitment, capacitation and evaluation were blinded to the kind of device they were providing to each subject. Patients and caregivers were also blinded to the kind of device they were using.

The primary outcome was the difference between the proportion of patients suffering any kind of adverse effects between groups during the whole trial. As secondary outcomes, cognition measured by the Alzheimer’s Disease Assessment Scale-Cognitive Subscale, Frontal Assessment Battery (FAB), semantic and phonetic Verbal Fluency Test (sVFT & pVFT respectively) and Montreal Cognitive Assessment (MoCA); daily living functionality, measured by the Katz Index of Independence in Activities of Daily Living (ADL) and the Lawton-Brody Instrumental Activities of Daily Living (iADL); and depression measured by the Geriatric Depression Scale Short Form (GDS-15). All secondary outcomes were measured by the researchers at baseline and six months after starting stimulation.

Baseline characteristics were reported as means and standard deviations. To assess differences between baseline characteristics in the sham and active FGMS groups; chi-squared or Mann-Whitney U test were performed, depending on the type of variable. The primary analysis was performed using an intention-to-treat approach, the proportion of patients suffering from any kind of adverse effect was compared between groups using a fisher test.

All secondary outcomes were analyzed with a one-way ANCOVA in which baseline scores were used as a covariate, post intervention scores as the dependent variable and the intervention (FGMS vs sham FGSM) as the independent variable. All statistical analyses were performed using IBM SPSS statistical software. Figures were designed with GraphPad prism for windows.

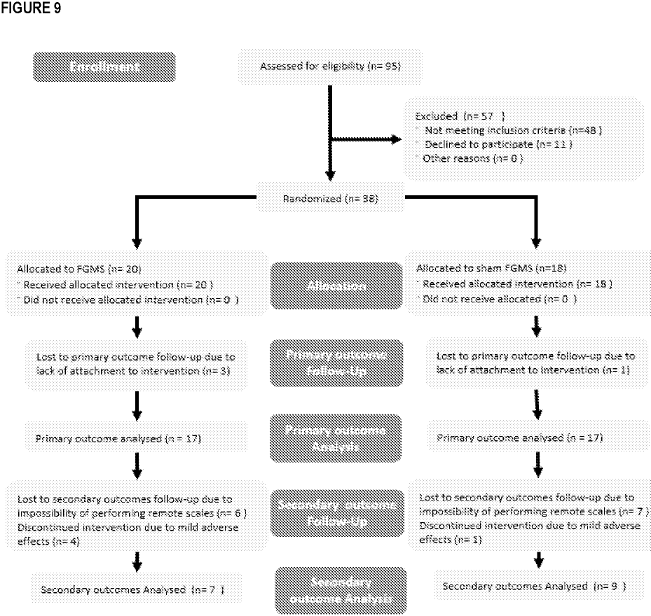

A total of 95 subjects were screened for eligibility; 38 met eligibility criteria and were randomized (see FIG. 9). A total of 20 subjects were allocated to the active FGMS group and 18 to the sham FGMS group. Four subjects (3 in the active FGMS and 1 in the sham FGMS) were excluded from the primary and secondary analysis due to lack of at least 80% of attachment to intervention. During the 6 month of the intervention, 4 subjects presented adverse effects in the FGMS group while just 1 subject presented adverse effects in the sham FGMS group. These 5 subjects were eliminated from the study once the adverse effects were reported and thus, were not included in secondary analysis. For the secondary analysis, 6 subjects from the active FGMS group and 7 subjects from the sham FGMS were lost due to impossibility of performing accurate remote evaluation for cognitive scales. In the end, 7 subjects from the active group and 9 subjects from the sham group were included for secondary outcome analysis.

Baseline demographic and clinical characteristics such as age, sex, baseline scores and relevant history of comorbidities were measured for the subjects included in primary analysis are described in FIG. 8. To evaluate differences in these variables, Mann-Whitney U test and chi-squared test were performed depending on the type of variable. Except for the prevalence of stroke, no statistically significant differences between groups at baseline variables were found.

Thirty-four patients, seventeen in each group, were analyzed for the primary outcome. Only 4 patients from the active FGMS group (23.52%) and one patient from the sham FGMS group (5.88%) presented any kind of adverse effects. Due to small sample size, Fisher’s exact test was run; there was not a statistically significant difference in proportions of 0.17, p = 0.335.

From the active FGMS group, the most common reported adverse event was sensory perception disturbances (3 subjects reported visual disturbances and 1 subject reported experiencing tinnitus), and headache (1 subject). From the sham FGMS group, one patient reported visual alterations that were ultimately related to a background condition. All adverse effects in the active FGMS were mild, transitory, and remitted without any medical intervention.

Regarding secondary outcomes, each variable was analyzed using a one-way ANCOVA to determine the effect of FGMS on post intervention scores. After adjusting for pre intervention scores, there was no statistical significant differences in ADAS-Cog score F(1, 13) = 0.790 , p = 0.390, partial η2 = 0.057 , FAB score F(1, 13) = 0.306, p = 0.590 , partial η2 = 0.023, sVFT score F(1, 13) = 0.250, p=0.627 η2 =0.022, pVFT score F(1, 13) = 0.821, p=0.384 η2 =0.069, MoCA score F(1, 13) = 0.002, p= 0.962 η2 =0.000, ADL F(1, 13) = 0.210, p=0.654 η2 =0.016, iADL score F(1, 13) = 3.017, p=0.106 η2 =0.188 and GDS-15 score F(1, 13) = 0.547, p=0.473 η2 =0.040. Due to the lack of statistical significance, post-hoc analyses were not performed. Analysis performed to the secondary outcomes are described in FIG. 9.

Regarding safety and usability, FGMS was adequately tolerated by most of the subjects. Only mild adverse effects were reported, and these effects remitted spontaneously without medical intervention. An important finding is that it is feasible to apply FGMS for a prolonged period of time (twice daily for 6 months) directly from the house of the patient. Non-invasive neuromodulation devices have shown promising results in treating neurodegenerative disorders; however, most of the current studies apply the neuromodulation intervention in a clinical setting for a limited period of time, which could affect real world efficacy and compliance for patients and caregivers. As only 3 subjects in the active FGMS and 1 in the sham FGMS did not complete the pre specified 80% of sessions, current results show that long term application of FGMS directly from home is feasible and has a high compliance rate.

In our sample, FGMS did not change the ADAS-Cog score after 6 months of daily application compared to sham stimulation. ADAS-Cog has been used for several years as the gold standard measurement of cognition in dementia populations; however, its utilization in subjects with MCI remains controversial, mainly because its ability to detect relevant changes in the initial stages of dementia has been discussed. Due to the small sample size, the possibility that ADAS-Cog was not sensitive enough to detect small changes in the studied population remains.

Other secondary outcomes were not modified by FGMS; cognitive measurements (FAB, MoCA, pVFT and sVFT) did not achieve statistically significant differences between groups. We choose several different measurements of cognitive functions to cover a broad range of clinical changes; interestingly, the FAB score showed a crossover interaction in which the effect of time on the score was opposite depending on the group the subject’s belonged to; unfortunately due to the small sample size, statistical significance was not achieved; further trials with adequate sample size focusing in frontal clinical changes could be regarded in a future, especially in dementia with frontal involvement such as frontotemporal dementia.

FGMS over the left prefrontal dorsolateral cortex applied twice a day for 6 months resulted to be a viable intervention that can be applied safely directly from home without supervision of a healthcare provider in an elderly population with MCI and mild Alzheimer’s disease. However, in this small sample, FGMS did not statistically significantly change cognitive, functionality or depression scores compared to sham stimulation.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A repetitive transcranial magnetic stimulation (rTMS) method for treating or preventing Alzheimer’s disease, comprising repetitively applying a magnetic pulse to the scalp of a patient in need thereof thereby stimulating neurons in the brain of the patient, wherein the magnetic pulse is applied:
repetitively over the patient’s brain; and
at a frequency of about 100 to about 150 Hz and an intensity of about 5,000 to about 15,000 milligauss.

2. A rTMS method for slowing or preventing a conversion of mild cognitive impairment (MCI) to Alzheimer’s disease, comprising repetitively applying a magnetic pulse to the scalp of a patient in need thereof thereby stimulating neurons in the brain of the patient, wherein the magnetic pulse is applied:
repetitively over the patient’s brain; and
at a frequency of about 100 to about 150 Hz and an intensity of about 5,000 to about 15,000 milligauss.

3. The method of either claim 1 or 2, wherein the magnetic pulse is applied at a frequency of about 125 to about 145 Hz.

4. The method of claim 3, wherein the magnetic pulse is applied at a frequency of about 135 Hz.

5. The method of any one of the previous claims, wherein the magnetic pulse is applied at an intensity of about 10,000 milligauss.

6. The method of any one of the previous claims, wherein the magnetic pulse generates an electric field of about 0.1 to about 10 V/m⁷.

7. The method of claim 6, wherein the magnetic pulse generates an electric field of about 0.5 to about 1.5 V/m⁷.

8. The method of claim 7, wherein the magnetic pulse generates an electric field of about 1 V/m⁷.

9. The method of any one of the previous claims, wherein the method is undertaken once, or twice, or thrice, or four times daily.

10. The method of claim 9, wherein the method is undertaken twice daily.

11. The method of any one of the previous claims, wherein the method is undertaken for greater than about 15 minutes.

12. The method of any one of the previous claims, wherein the method is undertaken for about 15 to about 60 minutes.

13. The method of claim 12, wherein the method is undertaken for about 30 minutes.

14. The method of any one of the previous claims, wherein the pulses are applied about 300 to about 400 times.

15. The method of claim 14, wherein the pulses are applied about 360 times.

16. The method of any one of the previous claims, wherein the pulses are applied discontinuously.

17. The method of any one of the previous claims, wherein the pulses last for about four seconds.

18. The method of claim 17, wherein the pulses last for about four seconds and followed by one second without pulsing.

19. The method of any one of the previous claims, wherein the treatment is applied chronically.

20. The method of any one of the previous claims, wherein the treatment is applied for greater than about 2 months.

21. The method of claim 20, wherein the treatment is applied for greater than about 6 months.

22. The method of claim 21, wherein the treatment is applied for greater than about one year.

23. The method of any one of the previous claims, wherein the treatment is self-applied.

24. The method of any one of the previous claims, wherein the magnetic pulse is applied using a device, the device being suitable for conducting electric current through a coil.

25. The method of claim 24, wherein the device generates a magnetic field.

26. The method of claim 25, wherein the device is suitable for home use.

27. The method of claim 26, wherein the device is portable.

28. The method of any one of the previous claims, wherein the patient is afflicted with MCI.

29. The method of claim 28, wherein the patient is afflicted with amnestic type MCI.

30. The method of claim 28, the patient is afflicted with non-amnestic type MCI.

31. The method of any one of claims 1-27, wherein the patient is afflicted with preclinical Alzheimer’s disease.

32. The method of any one of claims 1-27, wherein the patient is afflicted with mild Alzheimer’s disease.

33. The method of any one of claims 1-27, wherein the patient is afflicted with moderate Alzheimer’s disease.

34. The method of any one of claims 1-27, wherein the patient is afflicted with mild dementia.

35. The method of any one of the preceding claims, wherein the patient presents as having at least one biomarker indicative of AD, selected from high Aβ in cerebrospinal fluid, high Tau in cerebrospinal fluid, and the presence of the ApoE4 allele.

36. The method of any one of the preceding claims, wherein the patient presents as having a test score of one or more identified instruments for assessment of AD symptoms that is considered to indicate the presence of cognitive impairment or AD.

37. The method of claim 36, wherein the patient present as having a MoCA test score of less than about 26.

38. The method of claim 36, wherein the patient presents as having a ADAS-Cog test score of 2, 3, 4, or 5 on one or more subscores of the test.

39. The method of claim 36, wherein the patient presents as having a CDR score of at least about 0.5, at least about or 1, or at least about 2.

40. The method of any one of the preceding claims, wherein the method stimulates neurons around about 2 to about 3 cm from the skull.

41. The method of any one of the preceding claims, wherein the magnetic pulse is applied repetitively over the patient’s left prefrontal dorsolateral cortex.

42. The method of any one of the preceding claims, wherein the method stimulates neurons throughout the patient’s brain.

43. The method of any one of the preceding claims, wherein the method substantially stimulates neurons in the left hemisphere of the patient’s brain and/or frontal lobe of the patient’s brain.

44. The method of any one of the preceding claims, wherein the method substantially stimulates neurons in the cerebral cortex of the patient.

45. The method of any one of the preceding claims, wherein the method stimulates neurons outside of the patient’s left prefrontal dorsolateral cortex.

46. The method of any one of the preceding claims, wherein the method prevents or delays the progression of MCI to Alzheimer’s disease.

47. The method of any one of the preceding claims, wherein the treatment improves cognitive traits of the patient.

48. The method of any one of claims 1-46, wherein the treatment prevents diminution of cognitive traits of the patient.

49. The method of any one of the preceding claims, wherein the treatment slows a patient’s memory loss or retains or increases memory capacity, memory function, or cognitive function in the patient.

50. The method of claim 49, wherein memory capacity, memory function, cognitive function, or memory loss is assessed using one or more of the Instruments for assessment of AD symptoms listed in Figure 4.

51. The method of any one of the preceding claims, wherein memory capacity, memory function, cognitive function, or memory loss is assessed using the Montreal Cognitive Assessment (MoCA), Alzheimer’s Disease Assessment Scale-Cognitive Subscale (ADAS-Cog), Verbal fluidity test, Frontal Assessment Battery (FAB), Geriatric Depression Scale (GDS-15), Clinical dementia rating (CDR), EuroQoL-5D, Daily Life Activities of Katz (ABVD), Lawton Daily Life Instrumental Activities Scale (AIVD), and Bayer Scale of Activities of Daily Living (B-ADL).

52. The method of any one of the preceding claims, wherein an increase in the MoCA score or a decrease in ADS-Cog score following administration of rTMS indicates one or more of increased memory capacity, memory function, or cognitive function in the individual.

53. The method of any one of the preceding claims, wherein the treatment increases the patient’s MoCA score, as compared to the score at baseline.

54. The method of any one of the preceding claims, wherein the treatment decreases the patient’s ADAS-COG score, as compared to the score at baseline.

55. The method of any one of the preceding claims, wherein the treatment is used in tandem with one or more additional agents.

56. The method of any one of the preceding claims, wherein the treatment obviates the need for treatment with one or more additional agents.

57. The method of any one of the preceding claims, wherein the treatment is substantially free of adverse effects, optionally selected from epileptic seizures, nausea, and headache.
